(19) United States
(12) Patent Application Publication
(10) Pub. No.: US 2016/0081833 A1
(43) Pub. Date: Mar. 24, 2016

(54) INSTRUMENT INTENDED TO BE USED TO MODIFY THE VOLUME OF THE STOMACH OF A PATIENT

(71) Applicant: COUSIN BIOTECH, WERVICQ SUD (FR)

(72) Inventors: Jérome LEBLANC, COMINES (FR); Gilles SOLECKI, LANNOY (FR); Saloman BENCHETRIT, CALLUIRE ET CUIRE (FR)

(21) Appl. No.: 14/892,259

(22) PCT Filed: May 20, 2014
(86) PCT No.: PCT/FR2014/051171

§ 371 Date: Nov. 19, 2015

(30) Foreign Application Priority Data
May 20, 2013 (FR) 13 54507

(51) Int. Cl. A61F 5/00; A61B 17/04

(52) U.S. Cl. CPC A61F 5/0086; A61B 17/0482; A61B 2017/00818

(57) ABSTRACT

The invention relates to an instrument (10) intended to be used to modify the volume of the stomach of a patient.

The invention is characterized in that the instrument comprises:
a body (12) having a distal end (12a);
a clamping device (16) comprising first (18) and second (20) arms pivotably mounted at the distal end (12a) of the body (12) while pivoting relative to each other, each of the first and second arms (18, 20) comprising a plurality of guides for passage of a suture needle;
an opening device (30) for moving the first and second arms (18, 20) apart from each other by having them pivot relative to each other, said opening device comprising a spring member (32) connecting the first and second arms (18, 20) and tending to keep them apart from each other; and
a closing device (40) for bringing the first and second arms (18, 20) together by having them pivot relative to each other.

### BACKGROUND OF THE INVENTION

The present invention relates to the field of bariatric surgery, and more precisely operations aiming to modify the volume of the stomach of a patient suffering from obesity.

The invention relates more specifically to an instrument intended to be used to modify the volume of the stomach of a patient.

This instrument is therefore designed to carry out a gastroplasty operation by gastric plication and by laparoscopy. Gastric plication is a technique which consists of conferring restriction on the stomach without resection of the latter. It is generally done in laparoscopy by folding then suturing of the gastric wall by means of surgical instruments.

Existing surgical procedures which reduce the size of the stomach are many and diverse. One of the known techniques consists of performing gastrectomy by vertical stapling of the front and rear walls of the stomach, then resecting the part of the stomach now isolated from the gastric flow. This so-called longitudinal sleeve gastrectomy is a technique conducted with a linear stapler which then makes a cut between the two lines of staples. If this technique definitively removes the two tiers of the gastric volume by leaving a stomach modified in the form of a tube by way of vertical stapling over the entire height of the high gastric curvature, it has the disadvantage of showing fistulas which can cause serious complications.

Another surgical technique consists of performing so-called vertical and calibrated gastroplasty by the placing of a band. This technique, described in WO 2002/064041, consists of stapling the stomach in the extension of the oesophagus over a defined length then placing a band about the extension made to avoid its dilation. This technique needs the placing of a medical device formed by two plates articulated by a hinge which is implanted long-term in the body of the patient.

U.S. Pat. No. 5,549,621 describes a similar device. These two devices are very compressive and tend to cause necrosis of the tissue, resulting in fistulas.

These surgical techniques are called irreversible to the extent where they disallow a return to normal anatomy of the digestive system if the patient or the surgeon wants this.

To resolve these problems linked to the appearance of fistulas and the irreversibility of the operation, another significant gastroplasty technique invaginates the stomach reversibly and without risk of appearance of post-operative fistulas.

US 2012/0330329 describes a method of laparoscopic plication of the greater curvature of the stomach, performed by two lines of staples. The advantage of this operational technique is that it does not promote fistulas since there is no partial resection of the stomach. It is not however reversible because it would be very difficult, or even impossible, to pull out by laparoscopy the multitude of staples sometimes buried deeply in the gastric tissue.

US 2008/0319455 describes a method and medical instrumentation which carries out invagination of the stomach and the placing of a row of staples. This device dispenses with the problem of the appearance of fistulas but it is not reversible due to fixing of the fold by staples buried in the tissue.

### AIM AND SUMMARY OF THE INVENTION

A first object of the present invention is to propose an instrument intended to be used to modify the volume of the stomach of a patient which performs plication of the stomach substantially limiting the appearance of fistulas and being reversible.

The invention attains its aim by the fact that the instrument having a proximal end comprises:
a body having a distal end;
a clamping device comprising first and second arms pivotably mounted at the distal end of the body while pivoting relative to each other, each of the first and second arms comprising a plurality of guides for passage of a suture needle;
an opening device for moving apart the first and second arms from each other by having them pivot relative to each other, said opening device comprising a spring member connecting the first and second arms and tending to keep them apart from each other; and
a closing device for bringing the first and second arms together by having them pivot relative to each other.

The body has a longilineal form enabling its use in laparoscopy. The body has a longitudinal direction.

The first and second arms form a clamp which brings together two beads of the stomach wall when the instrument is brought to a closed position. The function of the guides arranged on the arms is to guide the suture needle, which easily sutures the beads, for example by overlock. The guides are advantageously arranged at regular intervals to ensure the continuity of invagination and reproducibility from one patient to another.

Advantageously, the instrument has an open position in which the first and second arms are moved apart from each other because of the opening device, and a closed position in which the first and second arms are brought towards each other because of the closing device, and the closing device has a locked position in which the instrument is kept in a closed position, and an unlocked position in which the instrument is free to be brought to an open position by the opening device.

Preferably, the closing device has several locked positions. In the locked position, the opening device cannot move the first and second arms apart from each other, whereas in the unlocked position the opening device is free to move the first and second arms apart from each other.

In other words, in locked position the instrument is kept in a closed position, whereas in unlocked position the instrument can be brought to an open position because of the opening device.

An aim is to improve the safety of the instrument by preventing accidental opening of the arms during suturing.

The instrument therefore has an open position in which the first and second arms are moved apart from each other, and a closed position in which the arms are brought towards each other to clamp the tissue to be sutured. Preferably, when the instrument is used empty, the first and second arms are arranged to extend parallel to the longitudinal direction of the body when the instrument is in closed position. Of course, during use the arms keep a certain spread between them due to the thickness of the tissue clamped in between the two arms.

It is understood that the closing device brings the instrument into a closed position, whereas the opening device brings the instrument into an open position.

In operation, to bring the instrument to the closed position the surgeon holds the body of the instrument and actuates the closing device in a first direction, for example by pushing it towards the distal end of the instrument. To bring the instrument to an open position the surgeon actuates the closing device in a second direction, opposite the first direction, after which the opening device moves the first and second arms apart from each other.

Preferably, the closing device is actuated by the surgeon, whereas the opening device acts automatically. In other words, without action by the surgeon the instrument tends to return to open position when the closing device is in unlocked position.

The spring member can be a cylindrical spring, a leaf spring or any other elastic device.

Advantageously, each of the first and second arms has an end pivotably attached at the distal end of the body.

Preferably, the ends of the first and second arms pivotably attached at the distal end of the body are pivotably mounted about the same axis of rotation. The instrument according to the invention is therefore simple and robust since there is a single pivot connection between the first arm, second arm and the body.

Advantageously, the closing device comprises an actuatable closing member cooperating with the first and second arms such that actuation of the closing member by the surgeon causes the first and second arms to move towards each other.

Preferably, the instrument is guided to the closed position by displacement of the closing member towards the distal end of the instrument.

More preferably, the body has a longitudinal direction and the closing member is slidably mounted on the body parallel to said longitudinal direction.

The body has at its proximal end a prehension portion allowing the surgeon to hold the instrument firmly during displacement of the closing member towards the distal end of the instrument.

According to a preferred embodiment, the closing device comprises an actuating arm which cooperates with a guide device mounted on the body allowing the actuating arm to slide according to the longitudinal direction of the body; the closing member is arranged at a distal end of the actuating arm and flanks the first and second arms such that displacement of the actuating arm towards the distal end of the instrument causes the first and second arms to move towards each other.

The actuating arm has for example the form of a rod whereof the distal end is fixedly attached to the closing member.

According to a variant, the guide device forms a slide. It can simply comprise a piece comprising two longitudinal grooves parallel to the longitudinal direction of the body. In this case, the closing member encloses the two arms so as to ensure holding of the actuating arm relative to the body.

Preferably, the proximal end of the actuating arm is provided with a prehension member to facilitate gripping and handling of said actuating arm. It could for example be a return when the actuating arm is a rod.

When the instrument is in open position, the first and second arms form a “V” whereof the tip is substantially at the distal end of the body. It is understood that the closing member, flanking the two arms, cooperates with the outer lateral faces of said arms. Also, displacement of the closing member towards the distal ends of the arms tends to reclose the “V” formed by the arms and, consequently, the effect is to bring the instrument to the closed position.

The closing member preferably comprises a piece in a U-shape having two wings facing each other which are arranged on either side of the assembly constituted by the first and second arms, by way of which the closing member flanks the two arms.

Preferably, the distance between the two wings is substantially equal to or slightly greater than the distance between the outer lateral faces of the two arms.

To limit displacement of the closing member towards the distal end of the instrument, the latter also comprises at least one stop member.

When the closing member is in contact with the stop member, the closing device is in locked position. In this locked position, the closing member, which flanks the two arms, prevents their moving apart and, consequently, ensures locking. Of course, when in use it is possible that the closing member cannot be brought into contact with the stop members due to the presence of tissue between the arms. However, the closing device will be in locked position because it opposes the arms being moved apart by the spring member.

Also, this stop member prevents the closing member from moving along the portions of the arms containing the guides.

Advantageously, the stop member projects from one of the first and second arms. Preferably, the stop member is arranged between the proximal end of the arm connected to the body and the guides.

Preferably, each of the first and second arms bears a stop member.

To make it easier to grip portions of walls, stomach or beads resulting from plications, at least the first arm advantageously comprises a hooking portion extending according to an inner lateral face of the first arm arranged facing an inner lateral face of the second arm.

This hooking portion can have a serrated form.

According to a preferred variant, the hooking portion comprises a plurality of barbs extending along the inner lateral face of the first arm.

Preferably, at least the second arm comprises orifices extending according to the inner lateral face of the second arm facing the inner lateral face of the first arm, said orifices being intended to receive the barbs of the first arm when the first and second arms are brought against each other. An aim of this configuration of the hooking portion is that the arms can be brought into contact against each other, or at the very least in direct proximity.

More preferably, each of the inner lateral faces of the first and second arms comprises a plurality of barbs and a plurality of orifices arranged to cooperate with the orifices and barbs formed in the other inner lateral face.

According to an advantageous embodiment, the guides terminate transversally on either side of each of the first and second arms.

Also, the guides are arranged such that, in closed position, the guides of the first arm are arranged facing the guides of the second arm so as to form a succession of continuous transversal passages which terminate on either side of the outer lateral faces of the first and second arms. This passage guides displacement of the needle according to the whole width of the instrument.

According to a preferred embodiment, the first and second arms have upper faces and in which the guides are slots terminating in the upper faces of the first and second arms.

Advantageously, the cross-section of the slots has a divergent form opening towards the upper faces. An aim is to enable easy removal of the instrument without the suture thread, especially put in place by overlock, hindering removal of the instrument.

This divergent form is preferably convex so as not to oppose removal of the thread out of the slot.

The invention also relates to a system intended to be used to modify the volume of the stomach of a patient, comprising an instrument according to the invention, a suture needle, and at least one notched suture thread whereof the notches form harpoons. An aim of this thread is to make for easier completion of the overlock and avoid the thread coming loose during suturing. In practice, one end of the thread comprises a loop to which the other end of the thread is introduced. The function of the harpoons is to enable displacement of the thread in the loop in a single direction only. According to the inventors, such a thread has never been used to perform an overlock in terms of a modification operation of the volume of the stomach.

Preferably, but non exclusively, the notched suture thread is of the V-Loc PBT No-absorbable type (registered trade mark).

A second object of the invention relates to a modification method of the volume of the stomach of a patient, wherein:
a portion of the fundus of the stomach is gripped; and
plication is performed by bringing said portion of the fundus towards the bottom of the stomach so as to form two beads extending substantially according to the greater curvature of the stomach.

By way of advantage:
an instrument according to the invention is provided;
the two beads are brought to each other by means of the instrument, by first placing the first and second arms on either side of the beads then actuating the instrument so as to bring together the first and second arms; then
an overlock is carried out by means of a suture needle and a suture thread so as to sutur together the two beads, the guides of the instrument guiding the passage of the suture needle during creation of the overlock.

Preferably, plication is carried out with a celioscopy clamp by pulling on the fundus or the large tuberosity of the stomach, after which invagination of the stomach forms naturally.

In keeping the stomach in this position by way of the celioscopy clamp, the two beads are clamped against each other by means of the instrument, achieving the overlock.

Preferably, three overlocks are performed to ligate the plication over the entire length of the beads.

### BRIEF DESCRIPTION OF THE DRAWINGS

The invention will be better understood from the following description of an embodiment of the invention given by way of non-limiting example, in reference to the appended drawing, in which:

FIG. 1 is a view perspective of the instrument in open position;

FIG. 2 is a detailed view of the distal end of the instrument in closed position;

FIG. 3 is an exploded view of the instrument according to the invention;

FIG. 3A is a view in longitudinal section of one of the slots of one of the arms;

FIG. 4 schematically illustrates the stomach of a patient;

FIG. 5 represents the plication step during which two beads form;

FIG. 6 represents the start of the suturing step conducted after the beads have been brought to each other by the instrument of FIG. 1;

FIG. 7 illustrates the end of the suturing step; and

FIGS. 8 and 9 illustrate in detail the system according to the invention and completion of the overlock by means of the notched thread.

### DETAILED DESCRIPTION OF THE INVENTION

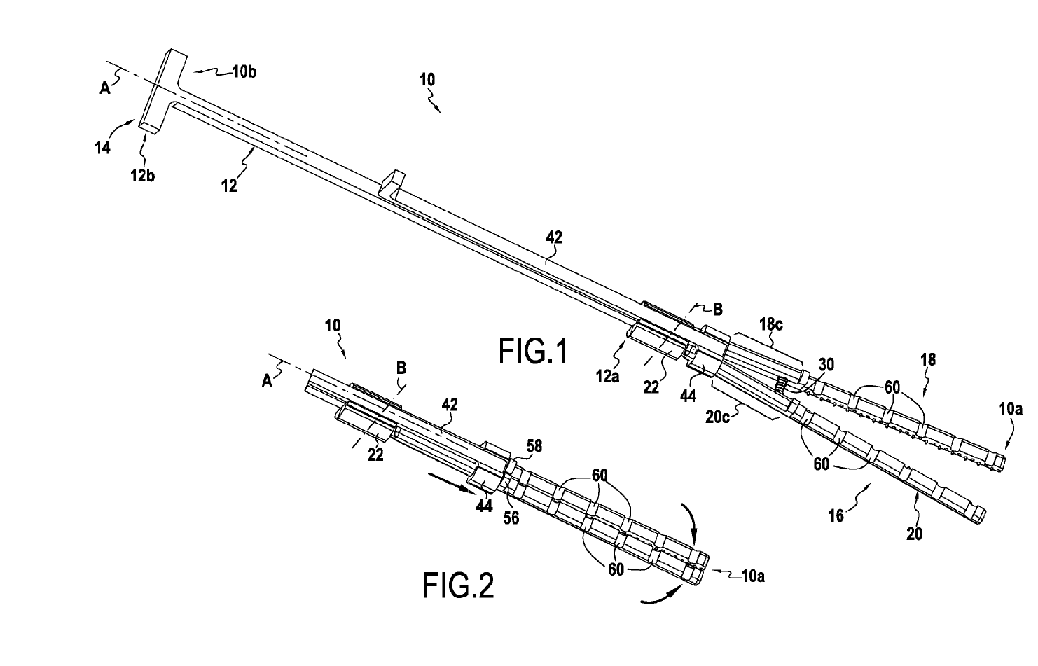

Based on FIGS. 1 to 3 a surgical instrument 10 according to the present invention will first be described. This instrument 10 is intended to be used to modify the volume of the stomach of a patient in terms of a bariatric surgery operation.

As visible from FIG. 1, the instrument 10 has a longilinear form and has a distal end 10a and a proximal end 10b. The expressions “distal” and “proximal” are defined in reference to the operator who manipulates the instrument 10 according to the invention.

The instrument 10 comprises a body 12 which has a distal end 12a and a proximal end 12b. It is clear that the proximal end 10b of the instrument 10 corresponds to the proximal end 12b of the body.

Also, the body is fitted at its proximal end 12b with a prehension device 14 having the form of a handle enabling the surgeon to firmly grip the instrument 10.

In this example, the body 12 has the form of a rod extending according to a longitudinal direction A. The instrument is dimensioned so that it can be introduced to the body of the patient by laparoscopy.

When said instrument is intended to be reused for several surgical interventions, the instrument is preferably, but not exclusively, made of metal alloy. It can be for example an ASI 302, an ASI 303, an ASI 304, an ASI 410, an ASI 416, an ASI 420, or the ASI 440 alloy, where ASI corresponds to the classification of the American Iron and Steel Institute (A.S.I.).

When the instrument must be disposed of after its use, it can be made of very rigid plastic material such as for example reinforced polymer of reference IXEF1032 (registered trade mark) of the company Solvay. The IXEF1032 (registered trade mark) is a polyarylamide reinforced by 60% glass fibre which has a high flexural modulus of the order of 21 GPa (made according to ISO 178). It can also be made with IXEF3008 (registered trade mark) which is a polyarylamide reinforced by 30% carbon fibre and which has a flexural modulus of the order of 23 GPa (made according to ISO 178).

At the end opposite the prehension device 14 the instrument 10 comprises a clamping device 16 which comprises a first arm 18 and a second arm 20 pivotably mounted relative to each other at the distal end 12a of the body 12.

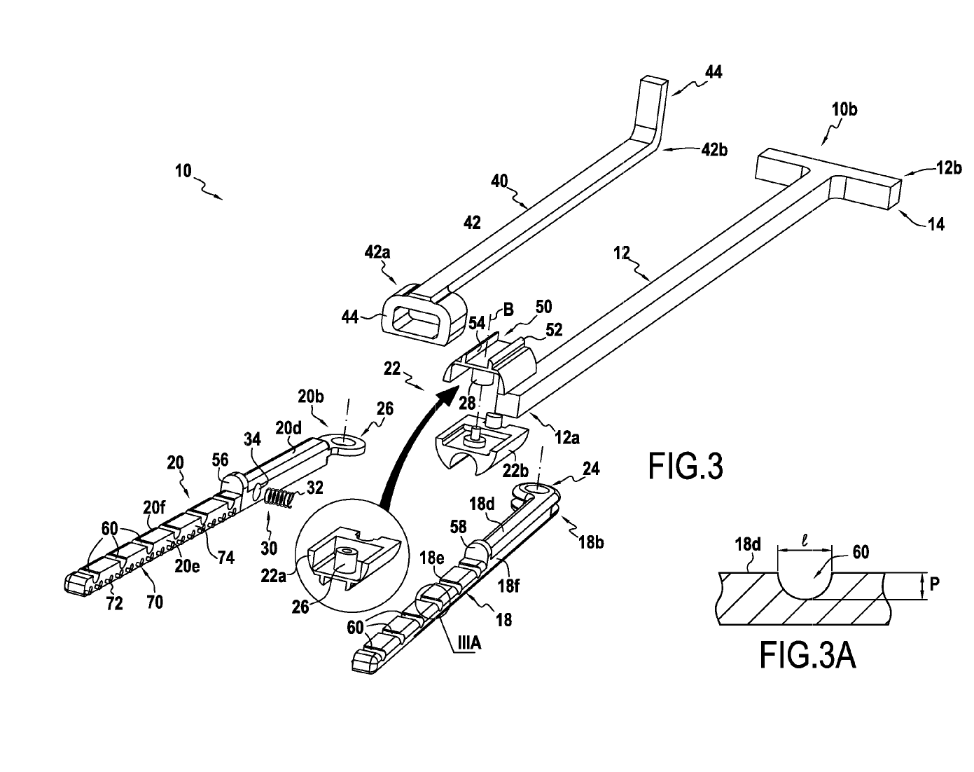

As visible from FIG. 3, each of the first and second arms 18, 20 has a proximal end 18b, 20b pivotably attached at the distal end 12a of the body 12. More precisely the ends 18b, 20b of the first and second arms 18, 20 which are pivotably attached at the distal end of the body 12, are pivotably mounted about the same axis of rotation B. The clamping device 16, comprising the first and second arms 18, 20, therefore forms a clamp.

To create the pivot connection (about the axis of rotation B) of the first and second arms 18, 20 relative to the body 12, the instrument 10 comprises in this example a connection piece 22 which is fixedly attached to the distal end 12a of the body 12. In the example of FIG. 3, the connection piece 22 comprises two pieces 22a, 22b fixed to each other while enclosing the distal end 12a of the body 12. The proximal ends 18b, 20b of the first and second arms 18, 20 comprise eyelets 24, 26 which are coaxial and which cooperate with a pivot axis 26 belonging to the connection piece 22.

The instrument 10 futher comprises an opening device 30 the function of which is to move the first and second arms 18, 20 apart from each other by having them pivot relative to each other about the axis B. In this example, the opening device 30 comprises a spring member 32 which joins the first and second arms 18, 20 by tending to keep them moved apart from each other. As is clear from FIG. 3, the spring member 32 is, in this example, a cylindrical spring which extends between the two arms and is housed in cavities 34 which are arranged in the inner lateral faces 18e, 20e of the arms 18, 20.

The instrument 10 further comprises a closing device 40 the function of which is to bring together the first and second arms 18, 20 by having them pivot relative to each other about the axis B.

In this example, the closing device 40 comprises an actuating arm 42 having a distal end 42a and a proximal end 42b. The actuating arm 42 is in the form of a rod whereof the proximal end 42b comprises a prehension member 44 in the form of an orthogonal return. The rod 42 extends according to the longitudinal direction A of the body 12.

The closing device comprises a closing member 44 fixed at the distal end 42a of the actuating arm 42. This closing member 44 is in the form of a sleeve. As is clear from FIGS. 1 and 2, the closing member 44 in the form of a sleeve is configured to enclose the assembly constituted by first and second arms 18, 20, and enable the sliding of the arms in the sleeve. So, actuation of the closing member by the surgeon causes the first and second arms to move towards each other.

More specifically, the closing member 44 in the form of a sleeve flanks the first and second arms 18, 20 such that displacement of the actuating arm 42 towards the distal end 10a of the instrument 10 causes the first and second arms 18, 20 to move towards each other. In other words, as illustrated in FIG. 2, actuation of the closing device by displacement of the actuating arm towards the distal end 10a of the instrument results in bringing the instrument 10 towards its closed position.

It is therefore understood that the closing member 44 is slidably mounted along the portions 18c, 20c of the first and second arms 18, 20 which are arranged near the distal end 12a of the body 12.

For this to happen, the closing member 44 is slidably mounted on the body 12 parallel to the longitudinal direction A of said body.

This sliding assembly is obtained because the actuating arm 42 cooperates with a guide device 50 which is mounted on the body so as to allow the actuating arm to slide according to the longitudinal direction A of the body. In this example the guide device 50 comprises two grooves 52, 54 extending parallel to the longitudinal direction of the body and projecting from the upper portion 22a of the connection piece 22. The guide device 50 forms a slide in which the actuating arm 42 slides.

In reference to FIG. 3, it is visible that each of the first and second arms 18, 20 further comprises a stop member 56, 58 configured to limit displacement of the closing member 44 towards the distal end 10a of the instrument 10. These stop members 56, 58 project from the upper lateral faces 18d, 20d of the first and second arms 18, 20.

In reference again to FIGS. 1 and 2 it is understood that displacement amplitude of the actuating arm 42 is between the connection piece 22 and the stop members 56, 58.

Given the above, it is understood that the instrument 10 has an open position, illustrated in FIG. 1, in which the first and second arms 18, 20 are moved apart from each other by the spring member 32, and a closed position, illustrated in FIG. 2, in which the first and second arms 18, 20 are positioned against each other by the closing member 44. During advance of the closing member towards the distal end 10a of the instrument 10 the arms are moved towards each other by going against the force exerted by the spring member 32.

In reference to FIG. 2, it is also clear that the closing device 40 has a locked position in which the instrument 10 is kept in closed position. In this locked position, the arms cannot move apart from each other.

When the closing member 44 is shifted towards the proximal end 10b of the instrument 10 so as to come into contact with the connection piece 22, the closing member 44 releases the pivoting of the first and second arms 18, 20 such that the spring member moves the arms apart from each other. In other words, in this unlocked position of the closing device the instrument is free to be guided to the open position by the opening device 30.

In reference again to FIG. 3 it is visible that each of the first and second arms 18, 20 comprises a plurality of guides 60. For each of the arms, these guides have the form of slots which terminate in the upper face 18d, 20d of the arms as well as in the inner 18e, 20e and outer lateral faces 18f, 20f of the arms. These slots 60 are arranged at regular intervals between the stop members and the distal ends of the arms. In this example, each of the arms comprises six slots.

As will be explained later, these guides in the form of slots are arranged to allow passage of a suture needle.

In reference to FIG. 2, it is understood that the slots 60 of the first and second arms 18, 20 are arranged facing so as to form continuous transversal passage for the needle. In this example the length of the slots corresponds substantially to the width of the arms, whereas the depth P of the slot is of the order of 5 millimetres, the width I of the slot being of the order of 3 millimetres.

In reference to FIG. 3A, it is visible that the cross-section of the slots has a divergent form opening towards the upper faces 18d, 20d of the arms. The aim of this form, in this case convex, allows easy withdrawal of the threads from the slots when the instrument is being withdrawn.

According to another aspect of the invention, each of the first and second arms comprises a hooking portion 70 extending according to the inner lateral face 18e, 20e of the arms. In this example, the hooking portion 70 comprises a plurality of barbs 72 which extend along the inner lateral faces of the first and second arms. These barbs 72 project from the inner lateral faces 18e, 20e of the arms so as to become hooked in the tissue. The arms also comprise orifices 74 which extend likewise according to inner lateral faces of the arms, these orifices 74 being intended to receive the barbs 72 when the first and second arms are brought against each other in closed position of the instrument 10.

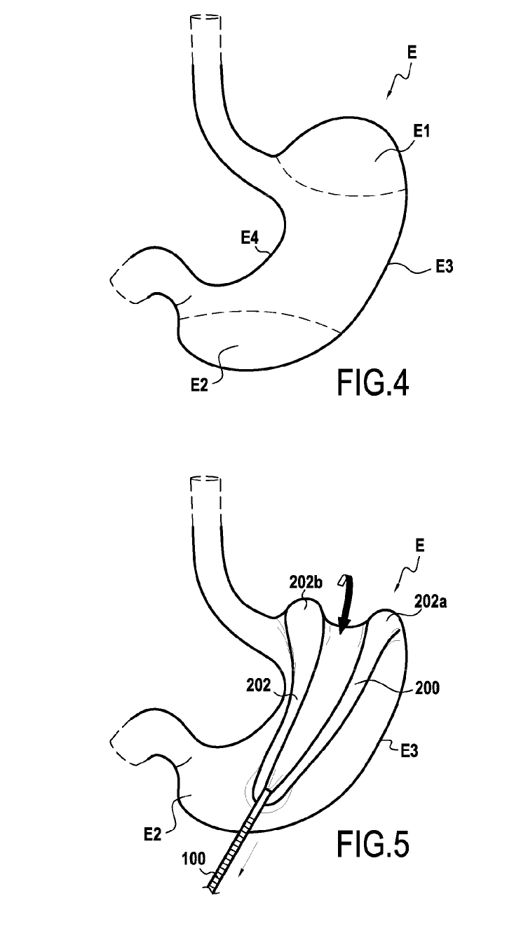

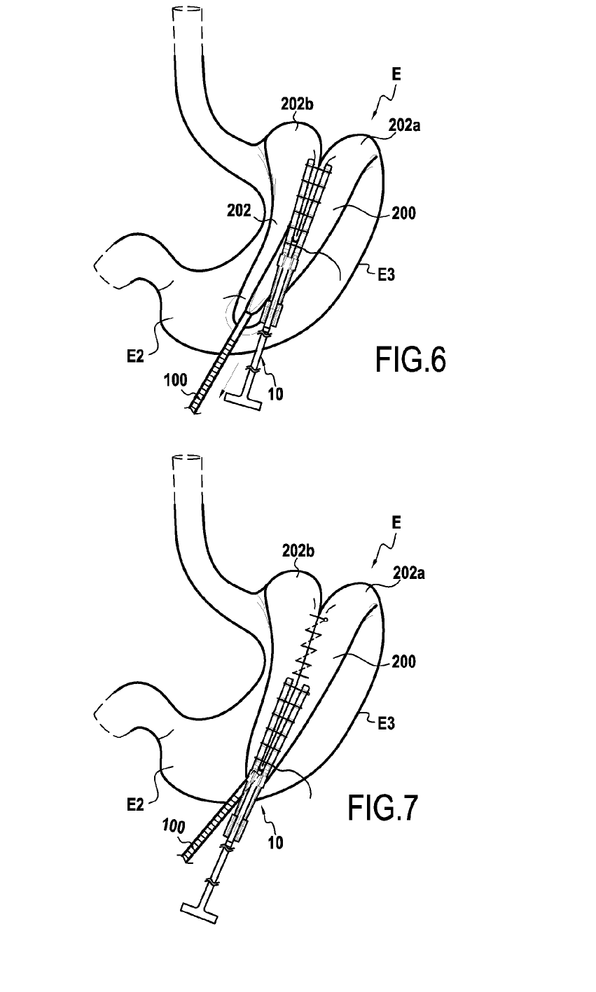

By way of FIGS. 4 to 7 an exemplary embodiment of a modification method of the volume of the stomach E of a patient will now be described. Conventionally, the stomach E comprises an upper part E1 generally called a fundus. The lower part of the stomach E2 is called bottom of the stomach E. The stomach E also has a greater curvature E3 and a lesser curvature E4, the lesser and greater curvatures E4, E3 being arranged between the fundus and the bottom of the stomach.

According to a first step of the invention, a portion of the fundus E1 of the stomach is gripped, for example by means of a celioscopy clamp 100. The portion of the fundus which has been gripped is guided, by means of the clamp 100, towards the bottom of the stomach, resulting in two beads 200, 202 forming, which extend substantially according to the greater curvature E3 of the stomach E. The creation of this plication is illustrated in FIG. 5.

Next, as the plication is maintained by means of the celioscopy clamp 100, the two beads 200, 202 are moved towards each other by means of the instrument 10 according to the invention. For this to happen, the upper parts 200a, 200b of the beads 200, 202 which are arranged near the fundus of the stomach are first brought together. The instrument 10 is brought to the open position near the bottom of the stomach, the first and second arms 18, 20 being arranged on either side of the beads 200 and 202. Then the closing device is actuated so as to bring together the first and second arms, which results in bringing the instrument into a closed position. Then a first overlock S1 is performed by means of a suture needle 300 and a suture thread 302. The overlock is made by successively passing the needle 300 through the slots 60 of the first and second arms so as to pass through the two beads, and in an end loop 404 of the thread 400 to form the first overlock S1, as illustrated in FIG. 9.

The thread 400 is notched. It comprises on its outer surface harpoons 402 which advantageously hold the suture thread clamped.

After the first overlock S1 has been completed, the instrument 10 is shifted towards the bottom of the stomach then actuated so as to bring together the median parts of the two beads 200, 202. The second overlock S2 is then done. Then the instrument 10 is again shifted and actuated so as to bring together upper parts of the beads 200, 202 to the suite from where a third overlock S3 is carried out.

Of course, without departing from the scope of the present invention a number of overlocks greater than three could be undertaken.

After the beads have been sutured by the three overlocks, the instrument 10 and the celioscopy clamp are withdrawn. This modification method of the volume of the stomach can be carried out very quickly, under one hour by an experienced surgeon.

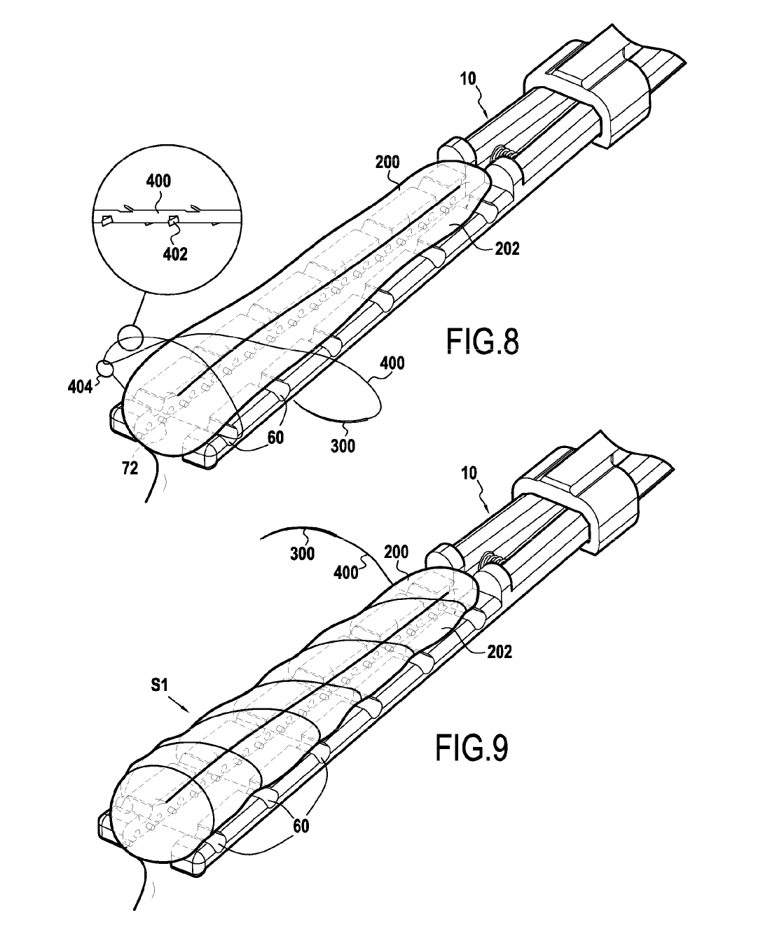

Also, FIGS. 8 to 9 illustrate a system S intended to be used to modify the volume of the stomach E of a patient, comprising an instrument 10 according to the invention, a suture needle 300 and at least one notched suture thread 400 the notches of which form harpoons.

1. An instrument intended to be used to modify the volume of the stomach of a patient, said instrument having a distal end and comprising:
a body having a distal end;
a clamping device comprising first and second arms pivotably mounted at the distal end of the body and enabling pivoting relative to each other, each of the first and second arms comprising a plurality of guides configured to permit passage of a suture needle;
an opening device configured to move the first and second arms apart from each other in a relative pivoting motion, said opening device comprising a spring member connecting the first and second arms and biasing the first and second arms away from each other; and
a closing device configured to pivot relatively the first and second arms towards each other.

2. The instrument according to claim 1, the instrument having an open position wherein the first and second arms are positioned apart from each other because of the opening device, and a closed position wherein the first and second arms are brought towards each other because of the closing device, and wherein the closing device has a locked position wherein the instrument is kept in the closed position, and an unlocked position wherein the instrument is free to be guided to the open position by the opening device.

3. The instrument according to claim 1, wherein each of the first and second arms has an end pivotably attached at the distal end of the body.

4. The instrument according to claim 3, wherein the ends of the first and second arms pivotably attached at the distal end of the body are pivotably mounted about the same axis of rotation.

5. The instrument according to claim 1, wherein the closing device comprises an actuatable closing member cooperating with the first and second arms such that the actuation of the closing member by the surgeon causes the first and second arms to move toward each other.

6. The instrument according to claim 5, wherein the body has a longitudinal axis, and wherein the closing member is slidably mounted on the body parallel to said longitudinal axis.

7. The instrument according to claim 6, wherein the closing device comprises an actuating arm which cooperates with a guide device mounted on the body allowing the actuating arm to slide along the longitudinal axis of the body, wherein the closing member is arranged at a distal end of the actuating arm and flanks the first and second arms such that displacement of the actuating arm towards the distal end of the instrument causes the first and second arms to move toward each other.

8. The instrument according to claim 6, further comprising at least one stop member configured to limit displacement of the closing member towards the distal end of the instrument.

9. The instrument according to claim 8, wherein the stop member projects from one of the first and second arms.

10. The instrument according to claim 1, wherein at least the first arm comprises a hooking portion extending according to an inner lateral face of the first arm arranged facing an inner lateral face of the second arm.

11. The instrument according to claim 10, wherein the hooking portion comprises a plurality of barbs extending along the inner lateral face of the first arm.

12. The instrument according to claim 11, wherein at least the second arm comprises orifices extending along the inner lateral face of the second arm facing the inner lateral face of the first arm, said orifices being configured to receive the barbs of the first arm when the first and second arms are brought against each other.

13. The instrument according to claim 1, wherein the guides terminate transversally on either side of each of the first and second arms.

14. The instrument according to claim 13, wherein the first and second arms have upper faces, and wherein the guides are slots terminating in the upper faces of the first and second arms.

15. The instrument according to claim 14, wherein a cross-section of the slots has a divergent shape opening towards the upper faces.

16. A system intended to be used to modify the volume of the stomach of a patient, comprising an instrument according to claim 1, a suture needle, and at least one notched suture thread, the notches forming harpoons.

17. A method for modification of the volume of the stomach of a patient, wherein:
a portion of the fundus of the stomach is gripped; and
plication is carried out by guiding said portion of the fundus towards the bottom of the stomach so as to form two beads extending substantially according to the greater curvature of the stomach.

18. The method according to claim 17, wherein:
an instrument having a distal end and comprising: a body having a distal end; a clamping device comprising first and second arms pivotably mounted at the distal end of the body and enabling pivoting relative to each other, each of the first and second arms comprising a plurality of guides configured to permit passage of a suture needle; an opening device configured to move the first and second arms apart from each other in a relative pivoting motion, said opening device comprising a spring member connecting the first and second arms and biasing the first and second arms away from each other; and a closing device configured to pivot relatively the first and second arms towards each other is provided;
the two beads are guided against each other by means of the instrument by first placing the first and second arms on either side of the beads then actuating the instrument so as to bring the first and second arms together; then
an overlock is carried out by means of a suture needle and a suture thread so as to sutur together the two beads, the guides of the instrument guiding the passage of the suture needle during creation of the overlock.
